(19) United States
(12) Patent Application Publication
(10) Pub. No.: US 2007/0059761 A1
(43) Pub. Date: Mar. 15, 2007

(54) Storage and use of colloids

(76) Inventor: Cynthia C. Bambad, Boston, MA (US)

Correspondence Address: JHK Law, PO Box 1078, La Canada, CA, 91012-1078, US

(21) Appl. No.: 10/557,009

(22) PCT Filed: May 13, 2004
(86) PCT No.: PCT/US04/15088

§ 371 Date: Nov. 14, 2005

Related U.S. Application Data
(60) Provisional application No. 60/469,979, filed on May 13, 2003.

(51) Int. Cl. G01N 33/53

(52) U.S. Cl. 435/7.1; 977/902

(57) ABSTRACT

The present invention discloses compositions and methods for enabling the longterm storage and/or use of colloid particles without substantial degradation of their performance, for example, by chemical or physical degradation, by particle aggregation, changes in pH and/or relative humidity, changes in salt concentration, and/or by a decrease in the binding activity or other detrimental change in biological, chemical, or physical properties of the colloid particles. In one aspect of the invention, the colloid particles are treated with an aggregation-preventing entity, for example, by immobilizing the entity relative to the colloid particle. In one embodiment, the aggregation-preventing entity forms at least a part of, and/or is immobilized relative to, a self-assembled monolayer (“SAM”) immobilized to the colloid particle. The aggregation-preventing entity may be added to non-aggregated or aggregated particles, for example to prevent aggregation and/or to reduce the degree of aggregation. In some embodiments of the invention, the colloid particles are essentially free of surfactants and/or other non immobilized aggregation-preventing entities. The colloid particles and/or solutions thereof may be stored before use without substantial degradation or aggregation over long periods of time in a dried state and/or at low temperatures. After storage, certain colloid particles provided by the invention can remain substantially non-aggregated. Various colloid particles of the invention may be used in many techniques, for example, in gels or other assay systems. In some cases, the colloid particles have a high degree of specificity and/or activity, which is due, at least in part, to their ability to remain in a substantially non-aggregated and detergent-free state during storage and/or use.

### BACKGROUND

1. Field of the Invention

This invention generally relates to colloids and, in particular, to the storage and use of substantially non-aggregated colloids and colloid-containing solutions.

2. Description of the Related Art

Colloid particles are used in many applications in chemistry and biology. For example, colloid particles have been disclosed for use in various diagnostic and biochemical assay techniques, including uses that enhance visibility and/or resolution of protein bands in SDS-PAGE gels, and for imaging purposes (see, for example, International Patent Publication Nos. WO 00/43791, published Jul. 27, 2000; and WO 00/43783, published Jul. 27, 2000). However, it is generally difficult to maintain colloid particles in solution without substantial and undesirable aggregation unless surfactants, detergents, or other chemical entities are added to the solution to prevent such aggregation. Colloid particles are typically stored refrigerated in a detergent-containing solution, which can prevent or inhibit particle aggregation. Removal of the detergent from the colloid solution can unmask surface charges on the particles and induce unwanted particle aggregation. This is unfortunate because the presence of detergents in biological or biochemical assays is often toxic or tends to interfere with diagnostics, or assays or other uses of the colloid particles.

While it is not uncommon to freeze or lyophilize chemical, biochemical, or biological species and reagents, it is generally not possible to freeze or lyophilize colloid solutions without substantial degradation of the utility of the solutions and the functionality of the colloid particles, due to particle aggregation. Thus, for example, directions accompanying many commercially-available colloid particle solutions specifically instruct the user not to freeze the solutions or expose them to temperatures less than 0° C.

### SUMMARY OF THE INVENTION

The invention relates generally to improvements in the ability to use and/or store colloid particles/nanoparticles in solution, or separate from solution. The invention permits extension of shelf-life, increases biocompatibility and makes shipping and storage more convenient. The subject matter of this application involves, in some cases, interrelated products, alternative solutions to a particular problem, and/or a plurality of different uses of a single system or article.

The invention, in certain aspects, is defined by a method. In one set of embodiments, the method includes a step of storing, for at least about one day, a plurality of colloid particles essentially free of a non-immobilized aggregation suppressor, such that the colloid particles remain substantially non-aggregated. The method includes, in another set of embodiments, a step of disaggregating aggregated colloid particles using a self-assembled monolayer-forming species. In yet another set of embodiments, the method includes a step of concentrating a solution containing a plurality of treated colloid particles, while maintaining the treated colloid particles in a substantially non-aggregated state in the solution, to a particle density that is greater than a density at which aggregation of identical but untreated colloid particles would occur without the presence of a non-immobilized aggregation suppressor.

In some embodiments, the method includes a step of freezing a solution containing a plurality of colloid particles such that the solution, when thawed, contains substantially non-aggregated colloid particles. In certain embodiments, the method includes a step of thawing a frozen solution containing colloid particles to recover substantially non-aggregated colloid particles.

In one set of embodiments, the method includes a step of drying a solution containing a plurality of colloid particles such that the solution, when reconstituted with solvent, contains substantially non-aggregated colloid particles. In some embodiments, the method includes a step of reconstituting dried colloid particles with solvent to recover substantially non-aggregated colloid particles.

In another aspect, the invention includes a kit. In one set of embodiments, the kit includes a solution comprising colloid particles, and instructions for storage of the solution at a temperature that does not exceed about 0° C. for at least about one hour. In some embodiments, the kit includes instructions for using an aggregation-preventing entity immobilized relative to at least one of a plurality of colloid particles.

Another aspect of the invention comprises an article. In one set of embodiments, the article includes a gel configured and adapted to facilitate separating molecules, the gel including a colloid particle at least partially coated with a self-assembled monolayer. In another set of embodiments, the article includes a gel configured and adapted to facilitate separating molecules, the gel including substantially non-aggregated colloid particles therein, the gel being essentially free of a non-immobilized aggregation suppressor.

The invention, in certain embodiments, includes a method having a step of detecting a protein or peptide in a gel using colloid particles having an aggregation-preventing entity immobilized relative to the colloid particles. In some cases, the method includes a step of detecting a protein or peptide in a gel using substantially non-aggregated colloid particles.

In another aspect, the invention is directed to methods of making any of the embodiments described herein. In yet another aspect, the invention is directed to methods of using any of the embodiments described herein.

Other advantages, novel features, and objects of the invention will become apparent from the following detailed description of non-limiting embodiments of the invention when considered in conjunction with the accompanying drawings, which are schematic and which are not intended to be drawn to scale. In the figures, each identical or nearly identical component that is illustrated in various figures typically is represented by a single numeral. For purposes of clarity, not every component is labeled in every figure, nor is every component of each embodiment of the invention necessarily shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

Non-limiting embodiments of the present invention will be described by way of example with reference to the accompanying drawings in which:

FIGS. 1A-1D are photocopies of photographs of colloids of the invention that remain in a substantially non-aggregated state in solution after freezing, compared to controls, according to one embodiment of the invention;

FIG. 2 is a bar graph displaying spectrophotometer measurements of colloids of the invention compared to controls, before and after freezing and freeze-drying.

FIGS. 3A-3B are photocopies of photographs of unmodified colloids attached to a gel, before and after freezing;

FIGS. 4A-4D are photocopies of photographs of colloids of one embodiment of the invention in a gel, before and after freezing;

FIGS. 5A-5B are photocopies of photographs of colloids of one embodiment of the invention in a gel, compared to unmodified colloids;

FIGS. 6A-6C are photocopies of photographs of colloids of one embodiment of the invention in a gel, showing a high degree of protein specificity; and

FIGS. 7A-7D are photocopies of photographs of colloids of an embodiment of the invention compared to controls, fresh and after freeze-drying.

### DETAILED DESCRIPTION

The present invention discloses compositions and methods for enabling the long-term storage and/or use of colloid particles without substantial degradation of their performance, for example, by chemical or physical degradation, by particle aggregation, changes in pH and/or relative humidity, changes in salt concentration, and/or by a decrease in the binding activity or other detrimental change in biological, chemical, or physical properties of the colloid particles. In one aspect of the invention, the colloid particles are treated with an aggregation-preventing entity, for example, by immobilizing the entity relative to the colloid particle. In one embodiment, the aggregation-preventing entity forms at least a part of, and/or is immobilized relative to, a self-assembled monolayer (“SAM”) immobilized to the colloid particle. The aggregation-preventing entity may be added to non-aggregated or aggregated particles, for example to prevent aggregation and/or to reduce the degree of aggregation. In some embodiments of the invention, the colloid particles are essentially free of surfactants and/or other non-immobilized aggregation-preventing entities. The colloid particles and/or solutions thereof may be stored before use without substantial degradation or aggregation over long periods of time in a dried state and/or at low temperatures. After storage, certain colloid particles provided by the invention can remain substantially non-aggregated. Various colloid particles of the invention may be used in many techniques, for example, in gels or other assay systems. In some cases, the colloid particles have a high degree of specificity and/or activity, which is due, at least in part, to their ability to remain in a substantially non-aggregated and detergent-free state during storage and/or use.

The following patent applications and publications are incorporated herein by reference: International Patent Application Serial No. PCT/US00/01997, filed Jan. 25, 2000, entitled “Rapid and Sensitive Detection of Aberrant Protein Aggregation in Neurodegenerative Diseases,” published as No. WO 00/43791; International Patent Application Serial No. PCT/US00/01504, filed Jan. 21, 2000, entitled “Assays involving Colloids and Non-Colloidal Structures,” published Jul. 27, 2000 as International Patent Publication No. WO 00/43783; U.S. patent application Ser. No. 09/631,818, filed Aug. 3, 2000, entitled “Rapid and Sensitive Detection of Protein Aggregation”; a U.S. patent application Ser. No. 60/248,865, filed Nov. 15, 2000, entitled “Endostatin-Like Angiogenesis Inhibition”; U.S. patent application Ser. No. 10/003,681 of the same title, filed Nov. 15, 2001; and U.S. patent application Ser. No. 09/996,069, filed Nov. 27, 2001, entitled “Diagnostic Tumor Markers, Drug Screening for Tumorigenesis Inhibition, and Compositions and Methods for Treatment of Cancer.” “Colloids” or “colloid particles,” as used herein, refers to very small, self-suspendable and/or fluid-suspendable particles, including those made of material that is, for example, inorganic or organic, polymeric, ceramic, semiconductor, metallic (e.g. gold or silver), non-metallic, crystalline, amorphous, or a combination of these. Typically, colloid particles used in accordance with the invention are nanoparticles having sizes on the order of nanometers, for example, less than 200 nm or 250 nm in cross section in any dimension, more typically less than 100 nm in cross section in any dimension, and in most cases are of about 2-30 nm in cross section. One class of colloids suitable for use in the invention is about 10-30 nm in cross section, and another is about 2-10 nm in cross section. As used herein, these terms include the definition commonly used in the field of biochemistry.

It is to be understood that the terms “solution” and “suspension,” as used herein in reference to those containing colloid particles, are used interchangeably as is typical by those of ordinary skill in the art. A description of a “suspension” as applied to colloid particles also applies to a colloid particle “solution,” and vice versa, unless specifically indicated to the contrary.

A “self-suspendable particle,” as used herein, is a particle that is of low enough size and/or mass that it will remain in suspension in a fluid (typically an aqueous solution), without assistance (e.g., without use of a magnetic field or stirring), for at least 1 hour. Other self-suspendable particles will remain in suspension, without assistance, for 5 hours, 1 day, 1 week, 1 month, 3 months, 1 year or indefinitely, in accordance with the invention.

The term “aggregate” (noun) means a plurality which is a significant, detectable percentage of particles or colloid particles immobilized with respect to each other, with or without an intermediate auxiliary entity between the particles. “Aggregate” (verb) or “aggregation” means the process of forming an aggregate (noun). Typically, for prior art colloid solutions or articles containing colloid particles, aggregation occurs spontaneously during storage of the particles under certain conditions, sometimes even in the presence of a surfactant. Similarly, the term “non-aggregated” refers to a colloid particle free of any substantial or stable attachment to any other particle or surface (including another colloid particle), for example, free of covalent binding, ionic binding, or various long-duration non-specific interactions with other surfaces and/or particles such that it is self-suspended in solution. A solution of “substantially non-aggregated” colloid particles as used herein has less than about 30% of particles that are in the aggregated state. In certain embodiments, the solution of substantially non-aggregated colloid particles can have less than about 20%, in other embodiments less than about 10%, in other embodiments less than about 5%, in other embodiments less than about 4%, in other embodiments less than about 3%, in other embodiments less than about 2%, in other embodiments less than about 1%, and in yet other embodiments less than a detectable amount of colloid particles that remain in aggregated state.

The term “binding” refers to the interaction between a corresponding pair of molecules or surfaces that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones and the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc. The term “binding partner” refers to a molecule that can undergo binding with a particular molecule. Biological binding partners are examples. For example, Protein A is a binding partner of the biological molecule IgG, and vice versa.

As used herein, a component that is “immobilized relative to” another component either is fastened to the other component or is indirectly fastened to the other component, e.g., by being fastened to a third component to which the other component also is fastened. For example, a colloid particle is immobilized relative to another colloid particle if a species fastened to the surface of the first colloid particle attaches to an entity, and a species on the surface of the second colloid particle attaches to the same entity, where the entity can be a single entity, a complex entity of multiple species, another particle, etc. In certain embodiments, a component that is immobilized relative to another component is immobilized using bonds that are stable, for example, in solution or suspension. In some embodiments, non-specific binding of a component to another component, where the components may easily separate due to solvent or thermal effects, is not preferred.

As used herein, “fastened to or adapted to be fastened to,” as used in the context of a species relative to another species or a species relative to a surface of an article (such as a colloid particle), or to a surface of an article relative to another surface, means that the species and/or surfaces are chemically or biochemically linked to or adapted to be linked to, respectively, each other via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a colloid particle, a binding species specifically biologically coupled to an antibody which is bound to a protein such as protein A, which is attached to a colloid particle, a binding species that forms a part of a molecule, which in turn is specifically biologically bound to a binding partner covalently fastened to a surface, etc. As another example, a moiety covalently linked to a thiol is adapted to be fastened to a gold colloid particle since thiols are able to bind gold covalently. Similarly, a species carrying a metal binding tag is adapted to be fastened to a surface (e.g., the surface of a colloid) that carries a molecule covalently attached to the surface (such as thiol/gold binding), which molecule also presents a chelate coordinating a metal. A species also is adapted to be fastened to a surface if a surface carries a particular nucleotide sequence, and the species includes a complementary nucleotide sequence.

“Specifically fastened” or “adapted to be specifically fastened” means a species is chemically or biochemically linked to or adapted to be linked to, respectively, another specimen or to a surface as described above with respect to the definition of “fastened to or adapted to be fastened,” but excluding essentially all non-specific binding. “Covalently fastened” means fastened via essentially nothing other than one or more covalent bonds. For example, a species that is attached to a carboxylate-presenting alkyl thiol by essentially nothing other than one or more covalent bonds, which is, in turn, fastened to the surface of a gold colloid particle, is covalently fastened to that surface.

“Affinity tag” is given its ordinary meaning in the art. Affinity tags include, for example, metal binding tags and streptavidin (in biotin/streptavidin binding). At various locations herein, specific affinity tags are described in connection with binding interactions. It is to be understood that the invention involves, in any embodiment employing an affinity tag, a series of individual embodiments each involving selection of any of the affinity tags described herein.

As used herein, the term “determining” generally refers to the analysis of a species, for example, quantitatively or qualitatively, or the detection of the presence or absence of the species. “Determining” may also refer to the analysis of an interaction between two or more species, for example, quantitatively or qualitatively, or by detecting the presence or absence of the interaction.

As used herein, the term “drying” is used to refer to processes that remove a liquid component (e.g., water) from, for example, a solution. Various techniques for drying a solution are well known. Non-limiting examples include lyophilization (freeze-drying), centrifugation, heating of the solution to induce evaporation and/or boiling, filtration, or exposing the solution to a desiccating environment, for example, an environment containing anhydrous calcium chloride, anhydrous calcium sulfate, phosphorous pentoxide, and the like. Similarly, “reconstitution” are processes where a liquid such as water is added to dry material to, for example, form a solution. Non-limiting examples of reconstituting techniques include adding water to form a solution (“rehydration”), or exposing a dry material solution to a vapor such that the vapor is absorbed to form a solution.

Surprisingly, the articles, methods, and kits provided according to certain embodiments of the invention allow for the storage and/or use of solutions and/or articles (e.g., powders, tablets, etc.) containing colloid particles that remain (or become, upon reconstitution to form a solution in the case of certain articles, e.g. dried tablets) substantially non-aggregated even when stored at low temperatures, high particle densities, high salt concentrations, different pHs, different relative humidities, under desiccated conditions and/or in detergent-free states for extended periods of time, for example, under conditions where it would be expected that typical prior art colloid particles would aggregate. Various storage conditions for certain embodiments of the inventive colloids can include, for example, storage in desiccating environments, storage in solutions having high salt concentrations, acidic and/or basic solutions, or conditions below the freezing point of water or other solvents of solutions that contain the colloid particles. For example, in one embodiment, the colloid particles of the invention can be maintained in storage below the freezing point of water, without significant degradation or undesirable aggregation of the colloid particles while in storage and/or upon reconstitution. In another example, the colloid particles of the invention can be maintained in a lyophilized state in storage for extended periods of time without significant degradation or undesirable aggregation of the colloid particles. In yet another example, the colloid particles of the invention can be maintained in a solution having a high salt concentration for extended periods of time without significant degradation or undesirable aggregation of the colloid particles. In still another example, the colloid particles of the invention can be maintained in a solution that is either very acidic or basic for extended periods of time without significant degradation or undesirable aggregation of the colloid particles.

In some embodiments, most or substantially all of the colloid particles remain free from aggregation over extended periods of storage and/or under particular storage conditions, as further described below. In some embodiments, the present invention allows for extended storage after the preparation of colloid particles prior to their use, without the need to add additional chemicals such as surfactants or other non-immobilized aggregation suppressors to inhibit degradation or aggregation of the colloid particles. In other embodiments, the present invention may allow for the storage of colloid particles in solution at high particle densities, without the need to add surfactants, preservatives, or the like. In still other embodiments, the present invention may allow substantially non-aggregated colloid particles to be used without the presence of significant amounts of surfactants, preservatives, or the like, for example, in a gel or an imaging assay.

In one embodiment, colloid particles are treated with an aggregation-preventing entity such that the colloid particles remain substantially non-aggregated during storage, for example, under conditions where, in the absence of the aggregation-preventing entity, it would be expected that the colloid particles would become aggregated during storage. In some cases, the aggregation-preventing entity may be immobilized or fastened, for instance covalently, relative to the colloid particle. As used herein, an “aggregation-preventing entity” or an “aggregation suppressor” is a molecule or other entity able to keep colloid particles in a substantially non-aggregated state after the colloid particles have been stored, for example, during cooling, freezing, drying, etc. In some instances, the colloid particles may at least partially aggregate during storage; however, the aggregation-preventing entity allows the colloid particles to revert to a substantially non-aggregated state after storage, for example, upon heating, reconstitution (e.g., in a buffer), dilution, neutralization, etc. Examples of aggregation-preventing entities include, but are not limited to, chemical species, biochemical species, and/or biological species (e.g., thiols, polymers, or certain proteins, such as lyoprotectant proteins).

In one embodiment, an immobilized aggregation-preventing entity forms a self-assembled monolayer on colloid particles. In some such embodiments, the aggregation-preventing entity is immobilized or fastened to the surface of colloid particles to at least partially cover the surface. In some embodiments, such particle coverage can involve greater than about 70% coverage, in some embodiments greater than about 80% coverage, in some embodiments greater than about 90% coverage, in some embodiments greater than about 95% coverage, in some embodiments greater than about 97% coverage, and in some embodiments greater than about 99% coverage. In certain embodiments, the immobilized aggregation-preventing entity may essentially completely cover the surface of the colloid particle. “Essentially completely cover” as used herein in this context, means that there is no portion of the surface of the colloid particles that is not covered by the SAM that is able to directly contact a species in solution (e.g., water). For example, in one embodiment, the surface of the colloid particles includes, across essentially its entirety, a SAM consisting essentially completely of non-naturally-occurring molecules (i.e. synthetic molecules). The aggregation-preventing entity may define a “protective layer” that may prevent or reduce aggregation of the colloid particles during storage.

In contrast, a “non-immobilized aggregation suppressor,” as used herein, is a substance generally able to prevent aggregation of the colloid particles during storage, but that is not immobilized or fastened to the colloid particle when the colloid particles are in solution. A non-immobilized aggregation suppressor may interfere with or adversely affect systems in which the colloid particles are used (for example, because the non-immobilized aggregation suppressor is toxic, or will interfere with diagnostics, assays, etc.). For example, a non-immobilized aggression suppressor may be a surfactant, a detergent, a zwitterion, a preservative, a cryoprotective agent such as dimethyl sulfoxide, an emulsifying agent such as a glyceride or a polysorbate, or a protein or a peptide such as casein or gelatin. As a specific example, the non-immobilized aggregation suppressor may be a surfactant such as TWEEN® 20 (ICI Americas Inc., Bridgewater, N.J., USA), for instance, at a concentration of about 0.133% or greater. In some embodiments of the invention, a solution containing the colloid particles of the invention is “essentially free” of a non-immobilized aggregation suppressor, such as a surfactant. “Essentially free,” as used in the above context, means that the non-immobilized aggregation suppressor is present in solution at a concentration that, by itself, is insufficient to prevent aggregation of the colloid particles during typical storage conditions and/or the above-described storage conditions enabled by the present invention.

A non-immobilized aggregation suppressor can be distinguished from an immobilized aggregation-preventing entity as follows. Rinsing colloid particles several times with a fluid in which the aggregation suppressor is soluble will readily remove most or all of a non-immobilized aggregation suppressor from the colloid particles, and may result in the precipitation or aggregation of the colloid particles. In contrast, immobilized aggregation suppressors according to the invention are distinguished from non-immobilized aggregation suppressors in that a substantial fraction (i.e., greater than about 70%) of the aggregation-suppressor molecules will remain immobilized with respect to the colloid particles after one or more of the above-described rinsing steps. For example, in certain particular embodiments, greater than 70%, 80%, 90%, 95%, 98%, 99%, or substantially all of immobilized aggregation suppressors can remain immobilized with respect to colloid particles after several rinsing steps in a fluid in which the aggregation suppressor is soluble.

In one aspect, the aggregation state of the colloid particles may be determined spectrometrically, for example, using optical techniques such as laser light scattering, fluorescence, or absorbance. For instance, in one set of embodiments, the aggregation state of the colloid particles may be readily determined by measuring absorbance at various wavelengths, such at about 530 nm and/or about 650 nm. Non-aggregated colloid particles in solution may cause the solution to appear generally pink-colored (i.e., a peak at about 530 nm), while aggregated colloid particles in solution may cause the solution to appear purple or blue colored (i.e., a peak at about 650 nm). Thus, by using techniques such as those familiar to those of ordinary skill in the art, the aggregation state of colloid particles may be determined by measuring the absorbance, for example using a spectrophotometer.

In one aspect, the aggregation state of the colloid particles and/or the stability of the inventive colloid particles in solution may be assessed by measuring the resistance of the particles to salt-induced aggregation and/or precipitation. As unmodified colloid particles such as gold colloid particles may have a slight negative surface charge, which under certain conditions allows the colloid particles to remain dispersed because of repulsive forces (e.g., London dispersion forces), when salt is added to such a solution containing unmodified colloid particles, the surface charges of the colloid particles may be masked, resulting in aggregation of the colloid particles. Additionally, in the absence of salt in solution, charges on the surfaces of the unmodified colloid particles can co-localize to induce a dipole, which may urge the colloid particles to aggregate, which is an undesirable result in many cases (for example, aggregated colloid particles cannot be used in many assays). Thus, unmodified colloid particles in solution are meta-stable, i.e., the stability of the colloid particles in solution may be altered by altering the concentration of salt in solution.

To inhibit particle aggregation, unmodified colloid particles are typically stored in solutions containing non-immobilized aggregation inhibitors such as detergents, surfactants, polymers or other inhibitors. Aggregation is inhibited by forces such as steric hindrance, charge interactions, or ionic interactions, depending on the composition of the inhibitor. The addition of salt to these solutions of unmodified colloid particles containing non-immobilized aggregation inhibitors typically induces colloid aggregation, as the salt molecules may compete with the non-immobilized inhibitor molecules for binding to the particle surface and/or sequester the non-immobilized inhibitor molecules, which may result in exposure of the colloid surfaces, causing induction of particle aggregation.

In contrast, the colloid particles of the invention is stable with respect to the concentration of salt in solution in many cases. Thus, the stability of articles of the invention, and/or the determination or detection of immobilized or non-immobilized aggregation suppressors, may be tested by determining the state of the aggregation of the colloid particles in solution in the presence of varying concentrations of salt as previously described, for example by measuring the absorbance around 530 and 650 nm.

In one set of embodiments, as mentioned above, the immobilized aggregation suppressor includes and/or is formed as a self-assembled monolayer. As used herein, the term “self-assembled monolayer” (SAM) refers to a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. Some of the methods that can be used to form a SAM are described in U.S. Pat. No. 5,620,850, which is hereby incorporated by reference. See also, for example, Laibinis, P. E., Hickman, J., Wrighton, M. S., Whitesides, G. M., Science, 245:845 (1989); Bain, C., Evall, J., Whitesides, G. M., J. Am. Chem. Soc., 111:7155-7164 (1989); Bain, C., Whitesides, G. M., J. Am. Chem. Soc., 111:7164-7175 (1989), each of which is incorporated herein by reference. Certain embodiments of the invention make use of self-assembled monolayers (SAMs) attached to surfaces of colloid particles, and articles including colloid particles. In one set of embodiments, as mentioned above, SAMs formed essentially completely of synthetic molecules essentially completely cover the surface of a colloid particle. “Synthetic molecule,” in this context, means a molecule that is not naturally occurring, rather, one synthesized under the direction of human or human-created or human-directed control. In some cases, the SAM can be made up of SAM-forming species that form close-packed SAMs at surfaces, and/or these species in combination with other species able to participate in a SAM. In some embodiments, some of the species that participate in the SAM include a functionality that binds, optionally covalently, to the surface, such as a thiol which will bind to a gold surface covalently. In one embodiment, the SAMs are cross-linked.

A self-assembled monolayer on a surface of a colloid particle, in accordance with the invention, may be comprised of a mixture of species (e.g. thiol species when the colloid has a gold surface) that can present (expose) essentially any chemical or biological functionality. For example, such species can include tri-ethylene glycol-terminated species (e.g. tri-ethylene glycol-terminated thiols) to resist non-specific adsorption, species with charged headgroups, such as carboxy-terminated thiols, to cause the colloids to repel each other and other species (e.g. thiols) terminating in a binding partner of an affinity tag, e.g. terminating in a chelate that can coordinate a metal such as nitrilotriacetic acid which, when in complex with nickel atoms, captures a metal binding tagged-species such as a histidine-tagged binding species. In some embodiments of the invention, a self-assembled monolayer is formed on gold or silver colloid particles. In one embodiment, an aggregation-preventing entity is or forms a part of a self-assembled monolayer.

In some embodiments, other molecules or entities may be bound to a component of a protective layer on a colloid particle (e.g., a nonimmobilized aggregation suppressor containing a protective layer of SAMs). Virtually any species can potentially be immobilized on a colloid by being bound to a component of such a SAM protective layer, for example, proteins, signaling entities, binding partners, or other species. For example, a SAM may be located on the surface of a colloid as a protective layer, and a variety of species may be attached to the SAM.

In certain embodiments of the invention, the “activity” of a species attached to a colloid particle (defined as the capacity of a colloid particle to immobilize or fasten a target species thereto after storage, relative to the capacity of the colloid particle to immobilize or fasten the species thereto that would occur immediately after preparation and absent storage), is within 70% to 80%, preferably at least 90%, more preferably at least 94%, more preferably at least 96%, more preferably at least 98%, more preferably at least 99%, and in certain embodiments, remains substantially unchanged. The target species may be any species that it is desired to be immobilized or fastened onto the colloid particle, for example, a binding partner, as previously discussed. In some embodiments, the target species is immobilized or fastened to another colloid particle. By using the techniques of the present invention, the binding activity of certain embodiments of colloid particles may be maintained approximately constant over long periods of time without significant degradation (e.g., within 90% or 95% of the initial activity). In some embodiments of the invention, the binding activity is substantially maintained (i.e., is at least 70%), even after repeated cycles of storage and use, such as repeated freezing and thawing, or concentration and dilution.

One method of determining the binding activity of a species immobilized or fastened to a colloid particle after storage, relative to before storage, is as follows. A target species and a solution of colloid particles having an immobilized binding partner to the target species are combined together in such a way that the target species is allowed to become immobilized or fastened to colloid particles. The degree of immobilization or fastening of that species (e.g., the relative amount of that species initially present in solution, which becomes immobilized relative to the colloid particles) can be determined by a variety of suitable techniques well known in the art. In one embodiment, such determination is readily made using techniques familiar to those skilled in the art. For example, following immobilization of the target species on the colloid particles, its cognate antibody (with attached signaling entity) is allowed to bind. Unbound antibody is washed away and the resultant signal can be measured to quantify the amount of colloid-immobilized target species. The degree of immobilization or fastening of the particles and the target species is then similarly determined after storage, and the ratio of the degree of immobilization, before and after storage, is used to determine the degradation or change (if any) of the binding activity.

It is an advantageous feature of certain embodiments of the invention that the inventive colloid particles can be stored in a variety of preparatory states in a substantially non-aggregated configuration. For example, the colloid particles can be prepared with an immobilized aggregation suppressor in accordance with the invention and stored for various times, at various temperatures and/or at various relative humidities and then, later, functionalized or used in an assay, for example, in an assay where the colloids particles are allowed to aggregate under certain conditions selected to facilitate desirable aggregation, e.g. particle aggregation due to binding events between species immobilized on different colloid sets. Colloids of the present invention carrying, for example, immobilized affinity tags can also be stored as previously described so that, after storage, they can readily be derivatized and used. Colloid particles according to one embodiment of the invention also can be stored in a derivatized state essentially ready for use. For example, the invention enables the preparation of colloid particles carrying immobilized chemical, biological or biochemical compounds of essentially any nature (for example, being or including signaling entities, proteins, antibodies, neurological disease fibril-forming species, candidate drugs, candidate drug targets, etc.), that do not, when the colloid particles are in solution, aggregate to an undesirable extent, for example, during storage (i.e., the colloid particles are able to remain in a substantially non-aggregated state during storage and/or until specific aggregation is induced and/or desirable).

Certain colloid particle solutions or articles of the invention may be stored for extended periods of time before use without unacceptable levels of undesirable aggregation and/or loss of activity. An acceptable length of time of storage corresponds to any substantial length of time where the colloid particles remain in a substantially non-aggregated state and/or where the function of the colloid particles (for example, the binding activity) is maintained to a desirable degree during and/or after storage. In one embodiment, the colloid particles of the invention can be stored overnight, or for about one day; in other embodiments, the colloid particles can be stored for several days, for example, about two or three days while remaining in a substantially non-aggregated state. In some embodiments, the colloid particles can be stored for even more extended periods of time (i.e., they remain substantially non-aggregated as previously defined) after storage, for example, about 1 week, about 1 month, about 2 months, about 3 months, about 6 months, or even about 1 year. Certain colloid particles of the invention can also potentially be stored without substantial particle aggregation for longer periods or essentially indefinitely.

In some embodiments, the colloid particles of the invention, or articles containing the colloid particles, may be advantageously stored at a desired and/or advantageous temperature without substantial or detectable aggregation of the colloid particles, including at temperatures less than room temperature (i.e., temperatures less than about 25° C.). In some embodiments of the invention, especially those wherein colloid particles are suspended in an aqueous solution, the colloid particles may be stored at a temperature that is less than the normal freezing point of water (0° C.). As used herein, “freezing the colloid particle” refers to the freezing of the solution or matrix containing the colloid particle. In some embodiments, the invention provides methods of storing the colloid particles at temperatures below typical room temperatures, such as in a refrigerator, a freezer or in a liquid nitrogen tank. In certain cases, the storage temperature of the colloid particles is less than the freezing point of the solution or matrix containing the colloid particles. For example, in some embodiments, the colloid particle solutions may be stored for extended periods of time in a refrigerator without substantial particle aggregation, for example, at a temperature of between about 4° C. to about 10° C. The storage temperature may also be, in some embodiments, less than about 0° C., less than about −4° C. in other embodiments, less than about −20° C., in other embodiments less than about −80° C., or in other embodiments less than about −196° C. Additionally, certain inventive colloid particle solutions can be advantageously stored under storage temperatures which do not necessarily need to be maintained constant. For example, storage temperature may fluctuate due to the nature of the refrigerator or freezer, or the article may be moved from one location to another during storage, for example, from a liquid nitrogen chamber to a freezer. Colloid particles provided in accordance with certain embodiments of the invention can be stored in solution in various embodiments at any of these temperatures for potentially any of the periods of time noted herein.

For embodiments where the colloid particles of the invention are stored in a frozen solution, the solution typically is thawed before use. Conditions for thawing such colloid particle solutions, which can maintain desirable levels of colloid function, are generally not critical. For example, in some embodiments, thawing conditions can include setting the solution or article containing the colloid particles on a counter at room temperature, or heating the solution or article within an incubator or a heated water bath (e.g., at a temperature of about 37° C. or about 60° C., etc.). The thawing conditions need only be selected so as to be able to warm the colloid particles to a useful operating temperature while preventing thermal degradation of the colloid particles or material(s) carried thereon (e.g., resulting in excessive loss of binding efficiency, decomposition, and/or undesirable aggregation). In some embodiments, the thawing conditions are selected so as to minimize the thermal stress on the colloids, and/or to prevent refreezing or recrystallization of the solution during the thawing process (i.e., conditions which might result in additional degradation or decomposition of the colloid particles and/or substances carried thereon are avoided). Suitable thawing conditions may be chosen by those of ordinary skill in the art with the benefit of the present disclosure by routine experimentation and optimization.

In certain embodiments of the invention, the colloid particles, or articles containing the colloid particles, may advantageously be stored under dried and/or desiccating conditions, for example, under relative humidities that are low, and/or in certain cases controlled, relative to the ambient environment. In certain embodiments of the invention, the colloid particles may be stored under relative humidities of less than about 20%, in certain embodiments less than about 10%, in certain embodiments less than about 5%, in certain embodiments less than about 3%, in certain embodiments less than about 1%, or in certain embodiments essentially 0% (i.e., an environment where there is no detectable water vapor present). Environments in which the relative humidity can be controlled at a specific level (i.e., at 0%) are well-known by those of ordinary skill in the art. For example, the environment may be controlled by exposing a contained atmosphere to anhydrous calcium chloride, anhydrous calcium sulfate, or phosphorous pentoxide. In certain embodiments, the atmosphere may be partially or completely removed (i.e., as in a vacuum), such that there is no detectable water vapor present. In some cases, a dry, inert atmosphere may also be used to blanket the colloid particles (i.e., the colloid particles can be maintained in a dry, nitrogen, carbon dioxide, helium, and/or argon environment).

For embodiments where the colloid particles of the invention are lyophilized or otherwise dried, the colloid particles may be reconstituted before use. Conditions for reconstituting the dried colloid particles to form a solution thereof, for example, to maintain desirable levels of colloid function, are not generally critical, and can be readily selected by those of ordinary skill in the art. For example, in certain embodiments of the invention, the reconstituting conditions can include adding a solvent, such as water or an aqueous solution (i.e., saline), to the dried colloid particles, or exposing the dried colloid particles to an environment having a vapor and/or relatively high relative humidity. The reconstituting conditions need only be selected so as to be able to reconstitute the colloid particles while preventing degradation, decomposition, or aggregation. In certain cases, the reconstitution to form solutions of the colloid particles may include forming an aqueous solution containing the colloid particles. In certain embodiments, the reconstituting conditions are selected so as to minimize stress on the colloids. Particular conditions may be chosen by those of ordinary skill in the art using only routine experimentation and optimization. In some cases, the colloid particles of the invention can be exposed to large changes in relative humidities, including multiple changes, without substantial aggregation.

In other embodiments of the invention, the inventive colloid particles, or articles containing the colloid particles, may be exposed to high concentrations of salt in solution, without substantial aggregation. Examples of salt solutions include sodium chloride, saline, potassium chloride, etc. In some cases, the concentration of salt that the inventive colloid particles are exposed to without causing aggregation may be at concentrations able to cause the aggregation of similar but unmodified colloid particles. In such embodiments, the high concentration solutions may be reconstituted upon the addition of water, dilute solutions of salt or buffer, etc. In certain embodiments, the reconstituting conditions are selected so as to minimize stress on the colloids. Particular conditions may be chosen by those of ordinary skill in the art using only routine experimentation and optimization.

In certain embodiments of the invention, the inventive colloid particles, or articles containing the colloid particles, may be exposed to non-neutral pH's, or substantial changes in pH, without substantial aggregation. For example, the inventive colloid particles may be exposed to strong acid or strong base, without substantial aggregation. As used herein, a “acid” is given its ordinary definition as used in chemistry. In some cases, an acid may have a pH of less than about 7, less than 5, less than 4, less than 3, or less than 2 pH units, depending on the strength of the acid. Similarly, a “base,” or an “alkaline” is given its ordinary definition as used in the field of chemistry. In some cases, the base or alkaline may have a pH of at least about 7, at least about 8, at least about 9, at least about 11, or at least about 12 pH units. A “non-neutral” or a “non-pH-neutral” composition is a composition that is either acidic or basic (i.e., the composition has a pH that is either greater than or less than 7, preferably by a significant amount, such as by at least 1 or 2 pH units). In such embodiments, the colloid solution may be reconstituted by neutralizing the pH. In certain instances, the reconstituting conditions are selected so as to minimize stress on the colloids. Particular conditions may be chosen by those of ordinary skill in the art using only routine experimentation and optimization.

The colloid particle solutions of the present invention, in some embodiments, can be made to undergo multiple cycles of heating and cooling, thawing and freezing, changes in salt concentration or pH, and/or drying and reconstitution, without significant loss of performance (e.g., wherein the colloid particles are able to remain substantially or completely non-aggregated and/or without a substantial or detectable change in binding activity). For example, in certain embodiments, the present invention enables a “stock” solution containing inventive colloid particles to be stored in a refrigerator or a freezer, which solution can be heated or thawed at various intervals to prepare aliquots of a working solution. The heating and cooling cycles may be repeated a number of times, for example, at least twice, at least three times, at least five times, at least ten times, at least fifteen times, at least twenty times, at least fifty times, at least one hundred times, or any other desired number of times while the particles remain in a substantially non-aggregated state and/or without a substantial loss of binding activity. In another set of embodiments, a “stock” solution or article containing inventive colloid particles may be stored under a desiccated atmosphere, such as an atmosphere where the relative humidity is relatively low (e.g., less than 10% relative humidity, less than 5% relative humidity, or essentially 0% relative humidity). Before use, the colloid particles may be removed from the desiccated environment to a normal (i.e., ambient) atmosphere. The exposure to desiccating and nondesiccating (e.g., ambient) conditions may be repeated any number of time, for example, at least twice, at least three times, at least five times, at least ten times, at least 15 times, at least 20 times, at least 50 times, at least 100 times, or any other desired number of times while the particles remain in a substantially non-aggregated state and/or without a substantial loss of binding activity. In yet another set of embodiments, the desiccated colloids can be stored at temperatures lower than the freezing point of water. In other embodiments, the colloid particles may be stored in concentrated salt solutions, acidic or basic solutions, etc.

In one set of embodiments, the colloid particle solutions or articles of the invention can be concentrated (i.e., the particle density may be increased), e.g. to facilitate convenient shipping and/or storage, while the particles are maintained in a substantially non-aggregated state. The colloid particle density of the solution or article may be increased by any suitable technique, for example, centrifugation, evaporation, lyophilization, filtration, reverse osmosis, electrophoresis, dialysis, and the like. Suitable conditions for these techniques, for example, for lyophilization, may be determined using the teachings herein by those of ordinary skill in the art via only routine experimentation and optimization. Other potentially suitable concentrating techniques are also known to those of ordinary skill in the art. In some cases, the particle density can be increased by a factor of at least about 10, 20, 50, 100, 1000, 10,000, or more, while the particles are maintained in a substantially non-aggregated state. In some embodiments, the concentration of particles may be increased to a density that is greater than is typically achievable using only a non-immobilized aggregation suppressor. In certain embodiments, colloid particle densities of at least about 0.3% (volume/volume) can be achieved, while the particles are maintained in a substantially non-aggregated state. In other embodiments, colloid particle densities of at least about 1% (volume/volume), about 5%, about 10%, about 20%, about 30%, about 40%, about 50%, about 60%, about 70%, about 80%, about 90%, about 95%, about 97%, or about 99% can be achieved, while the particles are maintained in a substantially non-aggregated state.

In certain embodiments, the colloid particle solutions or articles of the invention may be repeatedly concentrated and diluted, depending on the application, while the particles are maintained in a substantially non-aggregated state. For example, certain embodiments allow the concentration and dilution cycles to be repeated a number of times, for example, at least twice, at least three times, at least five times, at least ten times, at least fifteen times, at least twenty times, at least fifty times, at least one hundred times, or any other desired number of times while the particles are maintained in a substantially non-aggregated state. In some cases, the colloid particles may be used, while the particles are maintained in a substantially non-aggregated state, after multiple concentration/dilution and/or heating/cooling and/or drying/reconstitution cycles.

In some embodiments, an aggregate of colloid particles can be disaggregated using certain techniques of the present invention. Essentially any solution or article containing aggregated colloid particles may potentially be treated so that the aggregated colloid particles are disaggregated, for example, a surfactant-free or surfactant-containing solution having aggregated colloid particles therein, or a solution of conventional colloid particles that has been frozen, concentrated, dried, and/or exposed to high salt concentrations or non-neutral pHs can be disaggregated. In one set of embodiments, the aggregated colloid particles are exposed to aggregation-preventing entities provided according to certain embodiments of the invention, for example, by mixing a solution containing the aggregation-preventing entities of the invention with the solution of aggregated colloid particles after the solution has been thawed and/or reconstituted, or by adding a solution containing aggregation-preventing entities of the invention to the solution of colloid particles before thawing and/or reconstitution. For example, the aggregation-preventing entities may be a self-assembled monolayer or a self-assembled monolayer-forming species (e.g. as previously described). In some cases, a solution of aggregated colloid particles is exposed such that, when the solution thaws or is reconstituted, the colloid particles in solution are exposed to the aggregation-preventing entity. Exposure of the aggregated colloid particles to inventive aggregation-preventing entities may result in at least partial disaggregation and dissociation of the colloid particles, for example, in certain embodiments greater than 50% disaggregation, in other embodiments greater than 60% disaggregation, in other embodiments greater than 70% disaggregation, in other embodiments greater than 80% disaggregation, in other embodiments greater than 90% disaggregation, or in other embodiments greater than 95% disaggregation. In some cases, substantially all of the aggregated colloid particles may become disaggregated using the techniques of the invention. In another set of embodiments, exposure of aggregated colloid particles to aggregation-preventing entities of the invention can result in an increase in binding activity, for example, an increase at least of about 20%, at least of about 30%, or more compared to the activity of the aggregated colloid particles.

Various colloid particle solutions and articles of the invention may be used in a wide variety of techniques where the use of colloid particles is advantageous, desired, indicated, or necessary. Various techniques for which the inventive colloid particles are potentially useful will be apparent to those of ordinary skill in the art. Certain inventive colloids may be used, for example, as a tracking or sensing agent. For instance, in certain applications, the inventive colloids may be used for determining binding between various species, for determining the presence or a property of a species, for electrical measurements, and/or for imaging purposes. Chemicals and/or binding agents may be attached to the inventive colloid particles in certain such cases. In some embodiments, the colloid particles may be attached to a chemical species and/or a biological species or structure (e.g., a drug candidate, a peptide, a protein, a cell, a tissue specimen, an organelle) for tracking and detection purposes.

In one aspect, the colloid particles of the invention may be used in a biological assay, for example, in a gel used to analyze components such as proteins, nucleic acids, or other chemicals or biological molecules. For instance, the gel may be an agarose gel or a polyacrylamide gel. In one set of embodiments, the gel is used in a Western blot, a Southern blot, or an SDS-PAGE assay, for example, to detect or determine the presence and/or concentration of one or more proteins, peptides, and/or nucleic acids within a given sample. In one embodiment, a peptide, a protein, or a series of peptides and/or proteins are separated on the basis of a difference in at least one characteristic property (e.g., molecular weight and/or size) in a gel, and inventive colloid particles are used to detect and/or analyze the presence and/or concentration of the proteins or peptides within the gel. For instance, certain colloid particles of the invention can be modified so as to bind specifically or non-specifically to a molecule(s) of interest. In certain embodiments, there may be a high degree of specificity of binding of the colloid particles with the molecule(s) of interest. For example, the detection of a protein or other molecule(s) of interest may be more specifically detected by using a SAM-coated colloid that includes a biospecific entity and/or a member of a biological binding pair, for example, a polar headgroup able to recognize a certain class of proteins, an NTA-Ni headgroup able to recognize His-tagged proteins, an antibody able to recognize an antigen or a specific target protein, etc. Thus, in one embodiment, the invention includes a colloid-based Western blot, which, in some cases, may be simpler than a traditional Western blot (for example, by avoiding the use of antibodies that are labeled with a signaling entity).

In some cases, the molecular weight resolution of the various bands of material detected by a gel may be enhanced using certain colloid particles of the invention. The enhancement in molecular weight resolution according to the invention may result in a resolution that is higher than is achievable using unmodified colloid particles or conventional dyes. The enhanced molecular weight resolution may be due to, for example, the lack of substantial aggregation of the colloid particles, and/or the lack of surfactant or detergent associated with the colloid particles, as the surfactant or detergent may also adversely affect the protein, nucleic acid, or other molecule(s) of interest, or the binding of the colloids thereto, in the gel. In some cases, a molecular weight resolution of at least about 700 kDa, 500 kDa may be achieved using the colloid particles of the invention. In other cases, a molecular weight resolution of at least about 400 kDa, at least about 300 kDa, at least about 200 kDa, at least about 100 kDa, at least 1 kDa, at least about 100 Da at least about 50 Da, at least about 20 Da, or at least about 10 Da may achieved using the colloid particles of the invention.

A “kit” provided according to certain embodiments of the invention typically defines a package or packages including instructions and/or any one or a combination of the compositions, articles, particles, or solutions of the invention. Alternatively, the kits can include the composition, articles, particles, or solutions of the invention, in combination with instructions of any form such that one of ordinary skill in the art would clearly recognize that the instructions are to be associated with the solution. For example, in one embodiment, a kit provided by the invention includes instructions that allow a user to learn how to store and/or concentrate and/or dilute solutions of colloid particles of the invention, or inventive articles containing a solution of colloid particles, under the inventive conditions or achieving inventive results, as described above (e.g., storage of the particles within a freezer or in a desiccated or ambient environment). In another embodiment, an inventive kit includes instructions that allow a user to learn how to maintain the relative binding efficiency of a colloid particle in solution in storage over an extended period of time. In yet another embodiment, an inventive kit includes instructions that allow a user to learn how to use aggregation-preventing entities to disaggregate unmodified, aggregated colloids. The instructions can be printed on a separate piece of paper, directly on a container, on a label adhered to a container, on a box within which the container is stored or sold, or the like. In one embodiment, the instructions include a link to a web page on the Internet where detailed or updated instructions may be found.

The kits described herein may also contain one or more containers, which can contain compositions such as colloid particles, colloid particle solutions, articles containing the colloid particles and/or colloid particle solutions, aggregation-preventing entities, signaling entities, aqueous and/or organic solvents or solutions that colloid particles may be dissolved or suspended in, salts useful for preparing aqueous solutions of the colloid particles, biomolecules, and the like. The kits also may contain instructions for mixing, diluting, and/or administrating the colloids or colloid solutions. The kits also can include, in the same or separate containers, one or more solvents, preservatives, and/or diluents (e.g., normal saline (150 mM NaCl), DMF, substituted thiols, 5% dextrose, etc.). The kits may further include additional containers for mixing, diluting, rehydrating, reconstituting, thawing, and/or administering the components to a sample or to a patient. The composition(s) in the kit may be provided in liquid solutions or as dried powders. When the composition(s) is provided as a dry powder, the powder may be reconstituted by the addition of a suitable solvent, which may or may not be provided. Solution forms of the composition(s) may be concentrated (as previously described) or provided ready for use.

The function and advantage of these and other embodiments of the present invention will be more fully understood from the examples below. The following examples are intended to illustrate the features of some embodiments of the present invention, but do not exemplify the full scope of the invention.

### EXAMPLES

### Example 1

Colloids coated with NTA-SAMs can be prepared using techniques as described in one or more of the following documents, each incorporated herein by reference: International Patent Application Publication No. WO 00/43791, published Jul. 27, 2000, entitled “Rapid and Sensitive Detection of Aberrant Protein Aggregation in Neurodegenerative Diseases,” by C. Bamdad, et al.; International Patent Application Publication No. WO 00/43783, published Jul. 27, 2000, entitled “Assays involving Colloids and Non-Colloidal Structures,” by C. Bamdad, et al.; International Patent Application Publication No. WO 02/01230, published Jan. 3, 2002, entitled “Rapid and Sensitive Detection of Protein Aggregation,” by C. Bamdad, et al.; International Patent Application Publication No. WO 01/78709, published Oct. 25, 2001, entitled “Treatment of Neurodegenerative Disease,” by R. Bamdad, et al.; International Patent Application Publication No. WO 01/92277, published Dec. 6, 2001, entitled “Electroactive Surface-Confinable Molecules,” by C. Bamdad, et al.; International Patent Application Publication No. WO 02/37109, published May 10, 2002, entitled “Detection of Binding Species with Colloidal and Non-Colloidal Structures,” by C. Bamdad, et al.; International Patent Application Publication No. WO 02/01225, published Jan. 3, 2002, entitled “Tandem Signaling Assay,” by C. Bamdad, et al.; International Patent Application Publication No. WO 02/01228, published Jan. 3, 2002, entitled “Interaction of Colloid-Immobilized Species with Species on Non-Colloidal Structures,” by C. Bamdad, et al.; International Patent Application Publication No. WO 02/29411, published Apr. 11, 2002, entitled “Magnetic In Situ Dilution,” by C. Bamdad; International Patent Application Publication No. WO 02/28507, published Apr. 11, 2002, entitled “Electronic Detection of Interaction and Detection of Interaction Based on the Interruption of Flow,” by C. Bamdad; and International Patent Application Publication No. WO 02/061129, published Aug. 8, 2002, entitled “Oligonucleotide Identifier,” by C. Bamdad, et al.

In some cases, the colloids were prepared as follows. 1.5 ml of commercially available gold colloid (Auro Dye by Amersham) were pelleted by centrifugation in a microfuge on high for 10 minutes. The pellet was resuspended in 100 μL of the storage buffer (sodium citrate and Tween-20). 100 μL of a dimethyl formamide (DMF) solution containing 40 μM nitrilo tri-acetic acid (NTA)-thiol, 100 μM ferrocene-thiol, and 500 μM carboxy-terminated thiol was added (the ferrocene signaling entity is optional). Following a 3-hour incubation in the thiol solution, the colloids were pelleted and the supernatant discarded. The colloids were then incubated in 100 μL of 400 μM tri-ethylene glycol-terminated thiol in DMF for 2 minutes at 55° C., 2 minutes at 37° C., 1 minute at 55° C., 2 minutes at 37° C., then room temperature for 10 minutes. The colloids were then pelleted and 100 μL 100 mM NaCl phosphate buffer were added. The colloids were then diluted 1:1 with 180 μM NiSO₄ in colloid storage buffer.

### Example 2

In this example, the ability of gold colloids, which were first treated according to the invention with immobilized aggregation-preventing entities, e.g. coated with self-assembled monolayers, to remain in a non-aggregated state after a single freeze thaw cycle was compared to that of unmodified gold colloids. The aggregation state of the particles was determined by observing the color of the colloid solutions. Recall that solutions of gold colloids that are in a non-aggregated state appear pink, while solutions of gold colloids that are in an aggregated state appear purple or blue, where the degree of blue correlates to the degree of colloid aggregation.

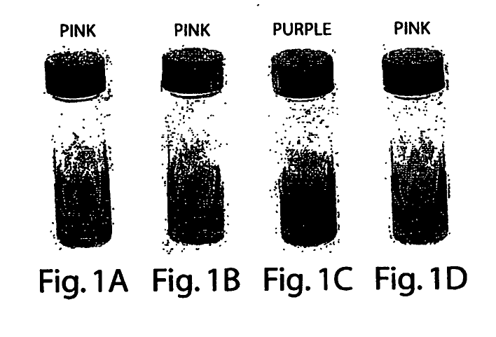

Unmodified gold colloid particles were purchased from Aurodye Forte (Amersham Biosciences, Piscataway, N.J.). Some of these colloids were coated with self-assembled monolayers (SAMs) comprising 2% nitrilotriacetic acid (NTA) terminated thiols, carboxy terminated thiols (about 80%) and ethylene glycol terminated thiols (about 18%), then resuspended in aqueous solution. Samples A and B of FIG. 1 are unmodified colloids and NTA-SAM coated colloids, respectively. Identical Samples C (unmodified colloids) and D (NTA-SAM coated colloids), also of FIG. 1, were frozen at −20° C. for 24 hours then thawed.

Samples A and B, which were not frozen and are shown as controls in FIG. 1. were light pink, indicating that the gold colloid particles in solution were substantially non-aggregated. Sample B, which was SAM-coated remained pink after freezing and thawing, indicating that the gold colloid particles in solution were substantially non-aggregated. In contrast, the solution in Sample C (unmodified colloids) was found to be a purple color, which indicated that the gold colloids in that sample have precipitated and formed aggregates, thus altering the color of the solution. Comparison of the inventive colloids (Sample D) and the unmodified colloids (Sample C) after freezing illustrated that self-assembled monolayers were able to prevent aggregation of the colloid particles during freezing and thawing.

### Example 3

In this example, unmodified colloids were compared to colloids prepared according to an embodiment of the invention after freezing and freeze-drying. The color of the resultant colloid solutions was quantified using standard techniques on a spectrophotometer. Peak heights at 650 nm (OD₆₅₀) (i.e., blue) were measured for fresh, previously frozen, or freeze-dried then reconstituted using both standard unmodified colloids and NTA-SAM-coated colloids.

The colloids in this example were prepared using techniques similar to those described in Example 1.

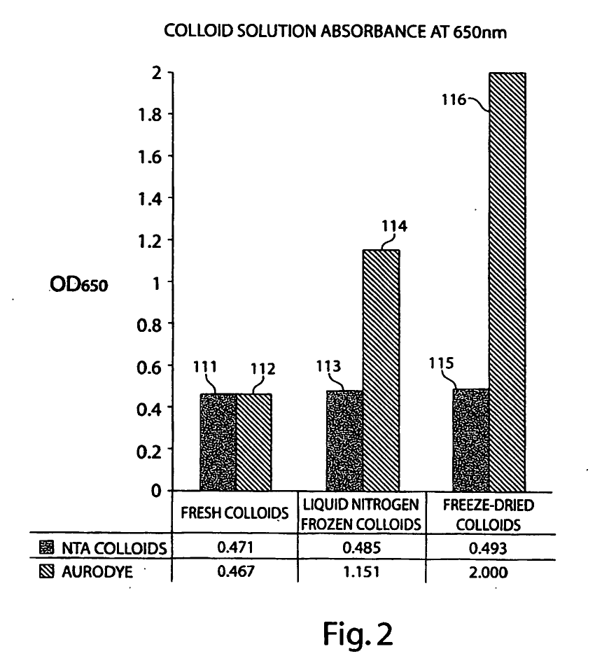

The bar graph of FIG. 2 shows that neither freezing nor freeze-drying NTA-SAM-coated colloids according to one embodiment of the invention resulted in significant changes to the colloids. For instance, a change of less than about 3% after freezing (113) and less than about 5% after freeze-drying (115) in the color of the colloid solution was measured at 650 nm (OD₆₅₀), compared to the NTA-SAM-colloids before freezing or freeze-drying (111). In contrast, freezing unmodified colloids caused the solutions to turn a purple/blue color, as shown by an increase of about 146% in the absorbance at 650 nm (114), relative to unfrozen, unmodified colloids (112). The change in color can indicate a high degree of colloid aggregation. Freeze-drying unmodified colloids may induce greater colloid aggregation, as shown by a larger increase in absorbance at 650 nm (116), relative to unfrozen, unmodified colloids (112).

### Example 4

This example demonstrates the stability of colloids according to an embodiment of the invention to remain in a substantially non-aggregated state after freezing or freeze-drying and thawing. This example further demonstrates the resistance of the inventive colloids to salt-induced aggregation over a wide range of salt concentrations.

The colloids in this example were prepared using techniques similar to those described in Example 1.

Table 1 demonstrates that after freezing or freeze-drying the colloids in saline solutions with salt concentrations ranging from 0-200 mM NaCl, there was at most a 5% change in the absorbance at 650 nm (i.e., blue) or 530 nm (i.e., pink).

TABLE 1

| | Absorbance at 530 nm | | | Absorbance at 650 nm | | |
|---|---|---|---|---|---|---|
| Reconstitution | Fresh | Liquid N₂ | Freeze-Dried | Fresh | Liquid N₂ | Freeze-Dried |
| buffer | Colloids | Frozen Colloids | Colloids | Colloids | Frozen Colloids | Colloids |
| 0 mM NaCl PO₄ | 4.024 | 3.933 | 3.885 | 0.477 | 0.483 | 0.469 |
| buffer | | | | | | |
| 100 mM NaCl | 4.037 | 3.824 | 3.899 | 0.474 | 0.485 | 0.480 |
| PO₄ buffer | | | | | | |
| 200 mM NaCl | 3.974 | 3.782 | 3.837 | 0.471 | 0.485 | 0.493 |
| PO₄ buffer | | | | | | |

Thus, in accordance with an embodiment of the invention, the colloids are able to remain in a substantially non-aggregated state after freezing or freeze-drying.

### Example 5

This example illustrates the use of inventive colloid particles in an SDS-PAGE experiment used to detect test proteins in a commercially available “protein ladder.”

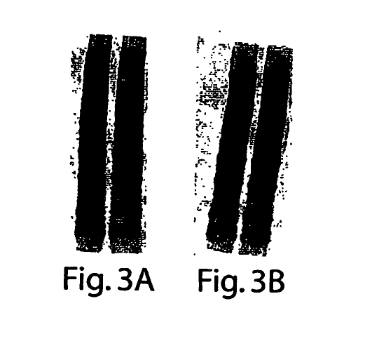

In FIG. 3, solutions containing unmodified gold colloid particles (i.e., gold colloid particles without any attached SAMs), before freezing (A) and after freezing (B), were used to stain a 5 μl protein Benchmark Ladder (Invitrogen; Carlsbad, Calif.) on a 1-12% SDS-PAGE gel. The bands corresponding to each protein in FIG. 3 were stained with the unmodified gold colloid particles using conventional methods. The unmodified colloid particles in FIG. 3, after freezing and thawing (B) illustrated a darker color and poor protein band molecular weight resolution, in comparison to the control experiment (A), thus indicating that aggregation of the gold colloid particles during frozen storage may have a detrimental effect on assays that use those colloid particles.

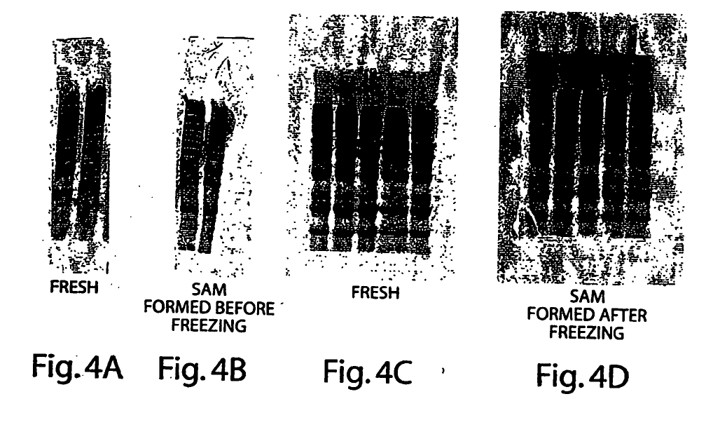

In contrast, in FIG. 4, gold colloid particles were coated according to an embodiment of the invention with 20 μM NTA-SAMs and were used to detect the test proteins in an SDS-PAGE experiment. In some cases, the SAMs were added to the gold colloid particles before freezing the particles (B), while in other cases, the SAMs were added after freezing and thawing of the solutions containing the colloid particles had occurred (D). These experiments were compared to control experiments where no freezing occurred (A and C). As can be seen in FIG. 4, the resolution of the protein ladders is comparable in both cases to their respective control experiments. Additionally, the experiments were highly reproducible from lane to lane in both color and resolution.

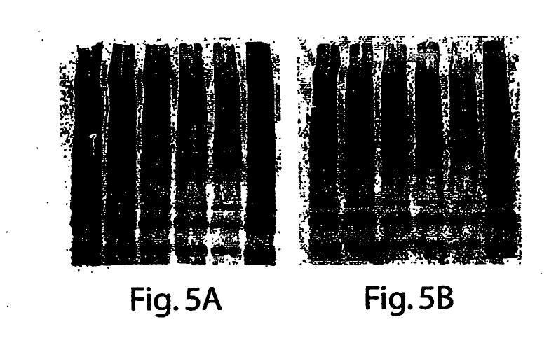

FIG. 5 illustrates the results of a comparison of unmodified gold colloid particles with surfactant (i.e., the control experiment) (A) with colloid particles coated with NTA-SAMs (B) as described in the present invention. The protein ladders used were 5 μl, 4 μl, 3 μl, 2 μl, 1 μl, and 5 μl protein Benchmark Ladders (Invitrogen). The resolution of the protein bands for the coated colloid particles was significantly higher than the unmodified gold colloid particles, indicating a higher degree of molecular weight resolution. Note, for example, the boxed regions, wherein a significant increase in resolution may be observed, as compared to the corresponding control experiment. The increase in resolution may be due, for example, to lower background binding of the gold colloid particles to the gel itself, and/or a decrease in the amount of protein denaturation due to the lack of surfactant in the SAM-coated colloid solution as compared to the controls.

This example therefore illustrates that while unmodified gold colloid particles typically aggregate during frozen storage, the inventive colloid particles coated with SAMs may not substantially aggregate during such storage if added before storage and, if added after storage, may cause substantial disaggregation of the colloid particles.

### Example 6

This example illustrates the specificity of certain colloid particles of the invention in resolving proteins of a cell lysate.

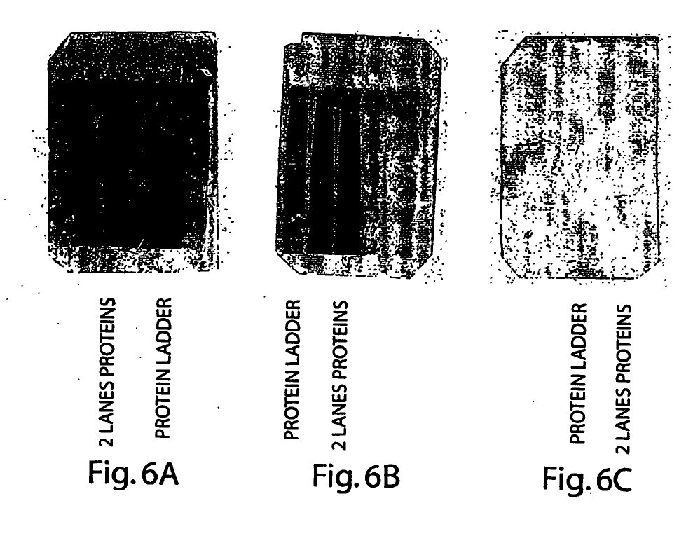

A cell suspension was lysed to produce a cell lysate. The cell lysate was analyzed in duplicate using SDS-PAGE (lanes 2 and 3 in the gels shown in FIG. 6), with a commercially available protein ladder as a control (lane 1). The lysate was stained using two sets of inventive colloids: (A) NTA-SAM-coated gold colloid particles, which specifically bind to proteins, and (B) C-16-COOH-SAM-coated gold colloid particles, which do not specifically bind to proteins.

As can be seen in FIG. 6, the NTA-SAM-coated colloid particles were found to have bound all of the proteins in the protein ladder (B), thus illustrating a high degree of specificity for protein and a pronounced lack of non-specific binding. Additionally, banding of the lysate lanes was also observed, indicating highly specific binding of the colloid particles to various cell lysate proteins. In contrast, the C-16-COOH-SAM-coated colloid particles did not significantly stain any of the proteins found within the cell lysates, or the protein ladders (C), thus indicating that the colloid particles in (C) did not display a significant degree of non-specific binding. As a control, the bare colloids (either, colloids not coated with SAM) did not show a high degree of specificity for individual protein bands (A).

Thus, this example illustrates a high degree of specificity and the low levels of nonspecific adhesion of these inventive colloid particles in resolving cell lysate proteins.

### Example 7

This example demonstrates the stability of colloids according to an embodiment of the invention to remain in a substantially non-aggregated state after freeze-drying.

In this example, the performance of commercially available gold colloids, (Aurodye Forte, Amersham Biosciences, Piscataway, N.J.) was studied, using fresh colloids and colloids that had been freeze-dried. Unmodified colloids and colloids prepared according to an embodiment of the invention were compared. The performance of the various colloids was determined using a protein gel staining experiment.

The protein gel was prepared as follows. Tris-HCl gels (Bio-Rad, Hercules, Calif.) were loaded with 5 microliters of Benchmark protein ladder (Invitrogen, Carlsbad, Calif.) and run at 100 V for approximately 90 min. Proteins were then transferred from gels to PVDF (polyvinyldifluorine) membranes (Millipore) via wet electrophoretic transfer in cold room (4° C.) overnight at 20 V. After transfer completion, the membranes were removed and incubated in PBS (phosphate-buffered saline) with 0.3% Tween-20 for 30 min at 37° C. The membranes were then washed at room temperature in PBS with 0.3% Tween-20 for 15 min, changing the buffer every 5 min. The membranes were then incubated in approximately 50 ml of the designated staining solution overnight. Blots were then washed in distilled water for 15 min and allowed to dry on filter paper.

Fresh, unmodified colloids (Aurodye Forte, Amersham, Piscataway, N.J.) were prepared as follows. The colloid particles as shipped from the supplier were stored in the original packaging and buffer at 4° C. until use. The staining solution was used as purchased (no dilution).

Freeze-dried, unmodified gold colloid particles were prepared as follows. 72 ml of unmodified colloid particles in their original storage buffer, which contains 0.133% Tween-20, were pelleted by centrifugation, and the supernatant was removed. The colloid pellet was then dried using a standard speed vacuum for 30 min at room temperature. The dried pellet was then frozen by placing the tube in liquid nitrogen for 30 min, then stored at −20° C. Before use, the colloid-containing tube was thawed at room temperature 30 min, then resuspended to their original concentration in 72 ml of the original colloid storage buffer. Resuspended colloids were used immediately.

NTA-SAM-coated colloids were prepared as follows. 6 ml of unmodified colloid particles in their original storage buffer, which contains 0.133% Tween-20, were pelleted by centrifugation, and the supernatant was removed. The colloid pellets were resuspended in 400 microliters of a DMF (dimethyl formamide) solution comprised of 2% NTA-thiol(HS—(CH₂)₁₁—(OCH₂CH₂)—OC(O)—NH—(CH₂)₄-nitrilo tri-acetic acid, 98% carboxy-terminated thiol(-[S—(CH₂)₁₁—COOH]) with a total thiol concentration of 1 mM and incubated for 2 hours. The colloid particles were then pelleted and resuspended in 200 microliters of the original Aurodye Forte storage buffer. To this solution was added a DMF solution containing 400 micromolar tri-ethylene-glycol-terminated thiol (C₁₁). The solution of colloids was then incubated at 55° C. for 2 min, 37° C. for 2 min, 55° C. for 1 min, then at 37° C. for 2 min. The colloids were then rested at room temperature for 15 min, then pelleted by centrifugation and resuspended in 400 microliters of PBS. 600 microliters of a NiSO₄ solution were next added (to complex the nitrilo tri-acetic acid with Ni⁺⁺) and incubated for 2 min, then pelleted and resuspended in 1 ml of PBS, at least twice to remove any residual storage buffer. The colloids were stored at 4° C. until use.

The freeze-dried NTA-SAM-coated colloids were prepared as follows. NTA-SAM-coated colloids were prepared as described above. The colloid particles were then pelleted by centrifugation, and the supernatant removed. The colloid pellet was then dried using a standard speed vacuum for 30 min at room temperature. The dried pellet was frozen by placing the tube in liquid nitrogen for 30 min, then stored at −20° C. Before use, the colloid-containing tube was thawed at room temperature 30 min, then resuspended to the original concentration in 6 ml of PBS. The resuspended colloids were used immediately.

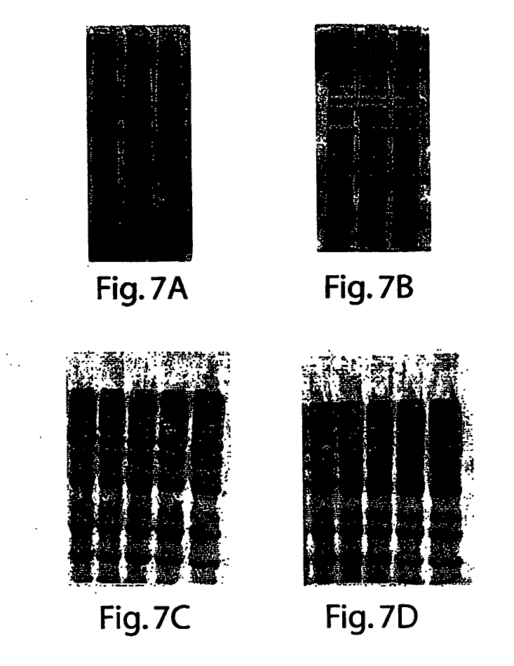

Example results from these experiments can be seen in FIG. 7. The gels pictured in FIG. 7C were stained with fresh NTA-SAM-coated colloids, while the gels pictured in FIG. 7D were stained with freeze-dried NTA-SAM-coated colloids. FIG. 7 illustrates that the performance of the inventive, SAM-coated colloids did not deteriorate (FIGS. 7C and 7D), in terms of protein band intensity, resolution or background staining, after freeze-drying and subsequent reconstitution. In contrast, the performance of the commercially available, unmodified colloids deteriorated after a single round of freeze-drying and thawing. For both fresh, unmodified colloid particles (FIG. 7A) and freeze-dried unmodified particles (FIG. 7B), the background of the gel was found to have been stained a darker purple/blue color, indicative of colloid precipitation. This shows that the unmodified colloid particles had reduced sensitivity and resolution to the gel stain.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” “composed of,” “made of,” “formed of” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, section 2111.03.

What is claimed is:

1. A method, comprising: storing, for at least about one day, a plurality of colloid particles essentially free of a non-immobilized aggregation suppressor, such that the colloid particles remain substantially non-aggregated.

2. The method of claim 1, wherein the colloid particles are in a solution.

3. The method of claim 2, wherein the solution is essentially free of surfactant.

4. The method of claim 1, wherein a chemical, biological, or biochemical entity is immobilized with respect to at least one colloid particle.

5. The method of claim 4, wherein the entity comprises an affinity tag.

6. The method of claim 1, wherein at least a portion of at least one of the colloid particles comprises a metal.

7-38. (canceled)

39. A method, comprising: thawing a frozen solution containing colloid particles to recover substantially non-aggregated colloid particles.

40. The method of claim 39, wherein an aggregation-preventing entity is immobilized relative to at least one of the colloid particles.

41. The method of claim 39, wherein at least one of the colloid particles comprises a self-assembled monolayer.

42. The method of claim 39, further comprising: adding an aggregation-preventing entity to the frozen solution.

43. The method of claim 39, further comprising, before thawing: freezing a solution to produce the frozen solution.

44. The method of claim 43, further comprising, before freezing: adding an aggregation-preventing entity to the solution.

45. The method of claim 43, further comprising, after thawing: adding an aggregation-preventing entity to the solution.

46. A method, comprising: reconstituting dried colloid particles with solvent to recover substantially non-aggregated colloid particles.

47. The method of claim 46, wherein an aggregation-preventing entity is immobilized relative to at least one of the colloid particles.

48. The method of claim 46, wherein at least one of the colloid particles comprises a self-assembled monolayer.

49. The method of claim 46, further comprising: adding an aggregation-preventing entity to the dried colloid particles.

50. The method of claim 46, further comprising, before reconstituting: drying a solution comprising colloid particles to produce dried colloid particles.

51. The method of claim 50, further comprising, before drying: adding an aggregation-preventing entity to the solution.

52. The method of claim 50, further comprising, after reconstituting: adding an aggregation-preventing entity to the solution.

53-73. (canceled)
